(19) United States
(12) Patent Application Publication
(10) Pub. No.: US 2018/0271392 A1
(43) Pub. Date: Sep. 27, 2018

(54) DEVICE AND METHOD OF USING HEXAXIAL ELECTROCARDIOGRAPH

(71) Applicant: Enrique Saldivar, Santee, CA (US)

(72) Inventor: Enrique Saldivar, Santee, CA (US)

(21) Appl. No.: 15/764,817

(22) PCT Filed: Oct. 4, 2016
(86) PCT No.: PCT/US2016/055290

§ 371 Date: Mar. 29, 2018

Related U.S. Application Data
(60) Provisional application No. 62/242,865, filed on Oct. 16, 2015.

(51) Int. Cl. A61B 5/0408; A61B 5/0404; A61B 5/044; A61B 5/0432; A61B 5/00

(52) U.S. Cl. CPC A61B 5/04085; A61B 5/0404; A61B 5/6828; A61B 5/04325; A61B 5/6824; A61B 5/044

(57) ABSTRACT

A device for obtaining an electrocardiogram of a user includes a contralateral arm electrode for independent contact with one of a left arm and a right arm of the user; an ipsilateral arm electrode for independent contact with an other of the left arm and the right arm of the user; a leg electrode for independent contact with a leg of the user; a controller configured to sense electric potentials between the left arm, the right arm, the left leg, and/or a common electric point, representative of a central terminal; and determine one, or multiple components, of a frontal plane, six-lead electrocardiogram of the user based on the resulting signals of the sensed electric potentials.

### FIELD OF THE INVENTION

The present invention relates to devices and methods to obtain an electrocardiographic signal, particularly to devices and methods to obtain a frontal plane, six-lead electrocardiogram.

### BACKGROUND OF THE INVENTION

Cardiovascular disease (CVD) is the most common cause of death around the world; accounting for 30% deaths worldwide, including 40% in high-income countries and 28% in low to middle-income countries. In 2001 CVD was responsible for 14% of the lost disability-adjusted years (DALYs) around the globe. By 2030, then the world population is expected to reach 8.2 billion and 33% of all deaths will be the result of CVD.

Within the population suffering CVD, a very important segment is the population suffering Ischemic Heart Disease (IHD). IHD is the leading cause of death and costs more than any other illness in the developed world. In the United States alone 13 million people have IHD. In the US approximately 650,000 patients experience a new Acute MI (AMI) and 450,000 patients experience a recurrent AMI each year.

The classification, diagnosis, and prognosis of Myocardial Infarction (MI) is based on the clinical presentation and the morphological features of the multiple-lead electrocardiogram (ECG); resulting in the classification of Non-ST-Segment Elevation MI (NSTEMI) and ST-Segment Elevation MI (STEMI). The prognosis of STEMI depends on its early detection. Most out-of-the-hospital deaths from STEMI are due to the onset of ventricular fibrillation. The majority of deaths due to ventricular fibrillation occur in the first 24 hours of the onset of symptoms; and more than half of the deaths occur in the first hour. During this crucial window of intervention, the greatest delay occurs not during transportation, but between the onset of pain and the patient's decision to call for help. These patients would benefit from having a readily available, wearable ECG. Additionally, it has been shown that Digital Health Interventions have a significant positive impact on risk factors for CVD.

Although there are some one-lead ECG wireless devices that provide portable and relatively non-intrusive solutions; these devices are incapable of evaluating STEMI and NSTEMI as its evaluation requires multiple ECG leads. Other multiple lead wirelesses ECG are bulky, have cumbersome wires for electrode connection, and are highly intrusive, preventing their daily use.

Therefore, currently there is an unmet need for an easy-to-use, readily-available device and method to provide patients and health care providers with a multiple lead ECG. Ideally this system would be with the patient at all times, have discreet usability, and provide crucial clinical information to the decision makers and health providers.

Significance of hexaxial approach: Measuring of the electric axis of the heart requires the hexaxial, or six lead, ECG. Additionally, it has been shown that the hexaxial ECG is efficient in the assessment of IHD, when compared to the current practice of 12-lead ECG; strongly suggesting that the implementation of a hexaxial ECG can overcome some of the serious current difficulties of wireless monitoring of the ECG. When compared to the current practice of 12-lead ECG, the hexaxial ECG has been shown to be efficient in the assessment of IHD; this evidence strongly suggests that the implementation of a hexaxial ECG can overcome some of the current technical difficulties in the wireless monitoring of IHD.

Other segments that would benefit from the availability of a hexaxial ECG: 1) Arrhythmias: 2.7-6.1 million people in the US have atrial fibrillation; 2) Hypertension: Almost 30% of the US adult population suffers hypertension, with an estimated total of 72 million people; 3) Obesity (presenting deviation of the electric axis or the heart): is calculated that nearly 79 million people in the US are obese, it is calculated that by 2025 20% of the global adult population will be obese; 4) Pregnancy (presenting deviation of the electric axis of the heart): Nearly 4 million women give birth in the US each year.

Clinical applications of hexaxial ECG include arrhythmia, conduction disturbances, myocardial ischemia, metabolic disturbances and high risk to sudden death. It is expected that the device and method of the present invention will benefit patients at risk of IHD, hypertension, patients with deviation of the electric axis of the heart, chronic pulmonary disease, and athletes. Except for arrhythmia, none of the above mentioned pathological conditions can be detected with the current one-lead ECG devices.

### SUMMARY OF THE INVENTION

An aspect of the invention involves a portable device and a method to record a hexaxial ECG using bipolar Leads (I, II and III) and unipolar leads of one's limbs (VR, VL and VF), or the bipolar Leads and the augmented unipolar leads of one's limbs (aVR, aVL and aVF). Lead I of the ECG is obtained when the electric potential generated by the heart is measured between the left arm (positive) and the right arm (negative). Lead II of the ECG is obtained by measuring the electric potential generated by the heart between the right arm (negative) and the left leg (positive). Lead III of the ECG is obtained when the electric potential generated by the heart is measured between the left arm (negative) and the left leg (positive). The bipolar leads I, II and III form an imaginary equilateral triangle, known as Einthoven's triangle. The representation of the voltage source of the scalar signal on each lead of the Einthoven's triangle can be expressed (using Kirchhoff's voltage law) as I-II+III=0, where the component of a given cardiac vector can be determined by projecting the vector along each side of the Einthoven's triangle. Reversing the process allows the determination of the cardiac vector. Additionally, unipolar leads of the frontal plane can be defined as signals obtained from more than one pair of electrodes; consisting of the measurement of the electric potential as detected on one electrode with respect to an equivalent reference electrode, consisting of the average of the signal captured on two or more electrodes. Of relevance, in ECG capturing is the determination of a reference electrical point to create the above mentioned equivalent reference electrode, by connecting each one or the limb electrodes (right arm, left arm and left leg) to equally valued resistors and connect them to a common node. This reference electrode is known as the Wilson's central terminal, which creates a central reference point. Wilson's central terminal=⅓(RA+LA+LL); where RA is the measurement obtained from the electrode placed on the right arm, LA is the measurement obtained from the electrode placed on the left arm, and LL is the measurement obtained from the electrode placed on the left leg. Wilson's central terminal corresponds to the average measurement of the limb electrodes. The electric potential between the right arm and Wilson's terminal is known as VR. The electric potential between the left arm and Wilson's terminal is known as VL, and the electric potential between the left leg and Wilson's terminal is known as VF. In each of the unipolar leads of the limbs (VR, VL and VF) the connecting resistance shunts the circuit between the central terminal and the limb electrode, resulting in a reduction of the amplitude of the measured electric signal. A strategy to ameliorate this signal attenuation is to disconnect the measured electrode from the central terminal and utilize a coupling resistor with half the resistance of the rest of the electrodes in the central terminal; the values, and the utilization, of the coupling resistors can vary depending on the electronic design. This strategy creates a central reference point, known as Goldberger's central terminal, which offers a 50% increase in the amplitude of the signal. The Goldberger's central terminal corresponds to the average measurement from the other two limb electrodes. Due to the increase in amplitude, these leads are known as augmented leads and are represented as aVR, aVL and aVF; aVR=RA−½(LA+LL); aVL=LA−½(RA+LL); and aVF=LL−½(RA+LA). The device includes surfaces with at least three easily accessible electrodes for the electric connection of the right upper limb, the left upper limb, and the lower left limb (or a region near the anatomical region to where these limbs attach to the thorax or the pelvis).

A further aspect of the invention involves a hexaxial electrocardiogram device for obtaining a frontal plane, six-lead electrocardiogram of a user includes a contralateral arm electrode for independent contact with one of a left arm and a right arm of the user; an ipsilateral arm electrode for independent contact with an other of the left arm and the right arm of the user; a leg electrode for independent contact with a leg of the user; an electronic circuit configured to: determine the electric activity of the heart and the magnitude and direction of its vector representation as measured between two points of the surface of the human body. The determination of the direction can be accomplished by the utilization of a differential amplifier, where the negative electrode is connected to the inverting input of the amplifier and the positive electrode is connected to the non-inverting input of the amplifier. With electrodes connected to the right arm, left arm and left leg of the subject sense lead I, by connecting the left arm to the positive input and the right arm to the negative input; sense lead II by connecting the right arm to the negative input and the left leg to the positive input; sense lead III by connecting the left arm to the negative input and the left leg to the positive; sense aVR (or VR) by connecting the right arm to the positive input and either Wilson's central terminal (for VR), or Goldberger's central terminal (for aVR), to the negative input; sense aVL (or VL) by connecting the left arm to the positive input and either Wilson's central terminal (for VL), or Goldberger's central terminal (for aVL) to the negative input; sense aVF (or VF) by connecting the left leg to the positive input and either Wilson's central terminal (for VF), or Goldberger's central terminal (for aVF) to the negative input; and determine a frontal plane, six-lead electrocardiogram of the subject based on the sensed signals.

One or more implementations of the aspects of the invention described above includes one or more of the following:

### BRIEF DESCRIPTION OF THE DRAWINGS

FIG. 1 is a perspective view of an embodiment of a hexaxial ECG recording device showing the device's casing with the strap and the location of a contralateral arm electrode, located on a face plate of the device, and an ipsilateral arm electrode, located on an inner surface of the strap;

FIG. 2 is a rear elevational view of the hexaxial ECG recording device of FIG. 1 and shows the position of a leg electrode on an outer surface of the strap;

FIG. 3 is a perspective view of a user wearing and using the hexaxial ECG recording device of FIGS. 1 and 2 shows the an example of the usability of the device, when worn on the left wrist. The figure shows the user wearing the hexaxial ECG recording device on his left wrist with the leg of his pants rolled up, to uncover the skin of the knee. The user is touching the contralateral arm electrode of the hexaxial ECG recording device with the right hand;

FIG. 4 is a perspective view of another embodiment of a hexaxial ECG recording device within a wristband, and shows the location of the contralateral arm electrode in this embodiment;

FIG. 5 is front elevational view of a further embodiment of a hexaxial ECG recording device within the form factor of a fob, and shows the relative position of the electrodes in this embodiment;

FIG. 6 is a perspective view of a still further embodiment of a hexaxial ECG recording device within the form factor of a wand, and shows the relative position of the electrodes in this embodiment;

FIG. 7 is a block diagram illustrating an example hexaxial ECG recording device according to the embodiments shown in FIGS. 1-6;

FIG. 8 is a block diagram illustrating an example wired or wireless processor enabled device that may be used in connection with various embodiments described herein;

FIG. 9 is a diagrammatic representation of an embodiment of an Einthoven's triangle and the connection of the electrodes according to the embodiments shown in FIGS. 1-7; and

FIG. 10 is a diagrammatic representation of an embodiment illustrating the connection of the electrodes to obtain a hexaxial ECG.

### DETAILED DESCRIPTION OF PREFERRED EMBODIMENT

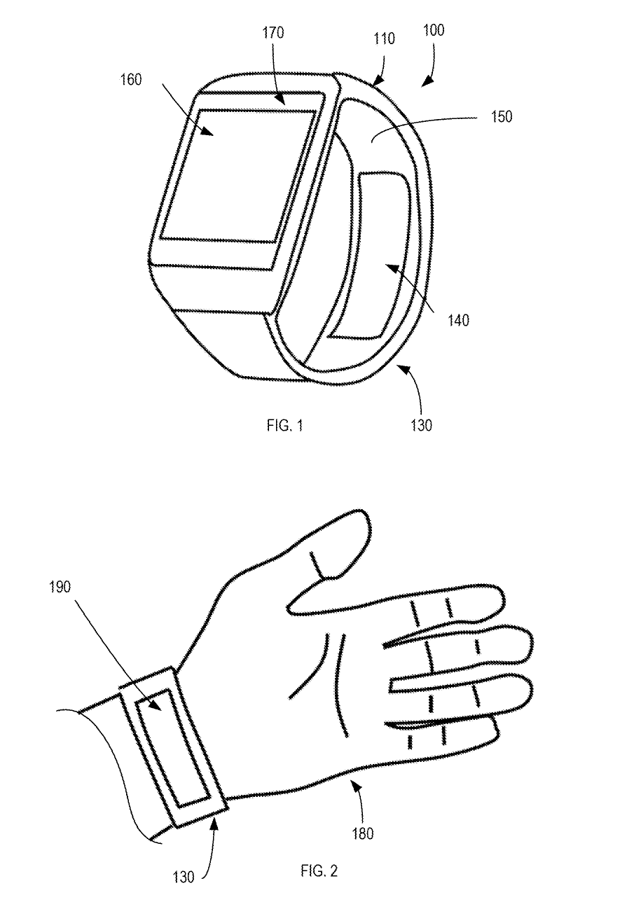

With reference initially to FIGS. 1-2, an embodiment of a portable hexaxial ECG recording device (“device”) 100 will be described. In this embodiment, components of the hexaxial ECG recording device 100 are incorporated into a wrist band 110. A casing, package, or housing 120, which is secured to a strap 130, packages electronic components of the device 100. An ipsilateral arm electrode 140 is located on an inner surface 150 of the strap 130. A contralateral arm electrode 160 is located on an external face 170 of the casing 120. With reference to FIG. 2, which shows the wrist band 110 affixed to a left hand 180 of the user, a leg electrode 190 is shown on an outer surface 200 of the strap 130. The leg electrode 190 is located anterior when the subject is in anatomical position. The electrodes 140, 160, 190 are connected to an electronic circuit inside the casing 120 through connecting cables embedded in the strap 130. In a preferred embodiment, the device 100 uses dry electrode technology. In an alternative embodiment, the device 100 utilizes contactless electrodes or other kind of electrodes.

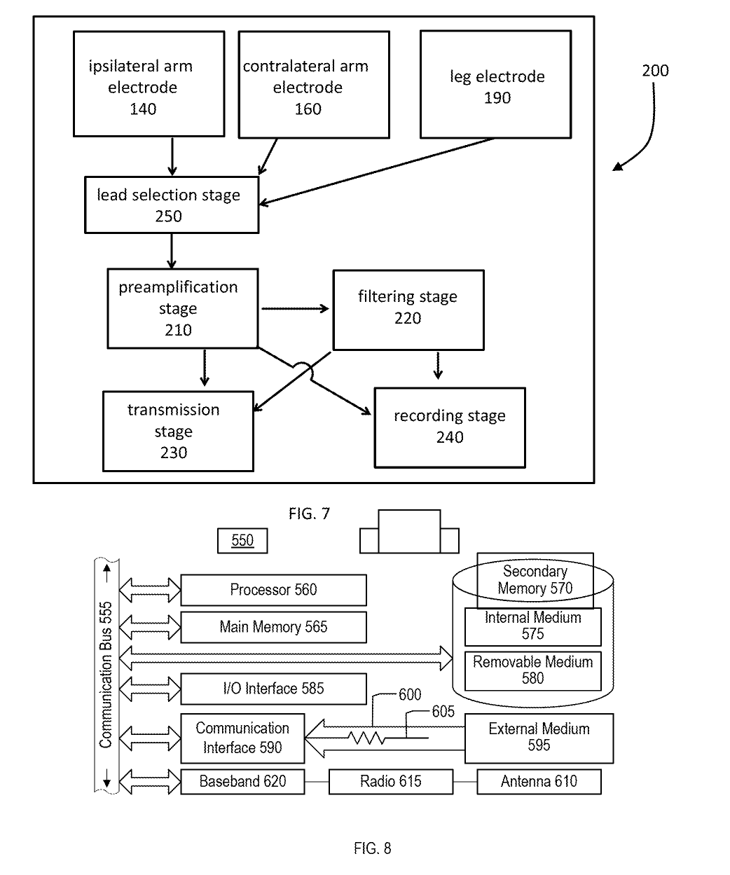

With reference to FIG. 7, a block diagram representing an electronic circuit 200 of the hexaxial ECG recording device 100 of FIGS. 1-2 will be described. The electronic circuit 200 includes the ipsilateral arm electrode 140, the contralateral arm electrode 160, and the leg electrode 190 that are part of electronic circuit 200. The electronic circuit 200 includes a preamplification stage or amplifier 210, a filtering stage 220, a transmission stage 230, a recording stage 240, and an optional lead selection stage 250. The optional lead selection stage 250 is composed of a set of controllable switches such as solid state relays or an array of single transistors. A proposed input and the preamplification stage 220 includes an instrumentation amplifier with basic functionality for common mode rejection. The filtering stage 220 includes a suitable band-pass filter for the ECG (e.g., in bandwidth of 0.5-150 Hz). The transmission stage 230 provides radio-transmission capabilities to send the captured ECG signal to another device. The recording stage 240 provides recording capabilities to record the captured ECG signal.

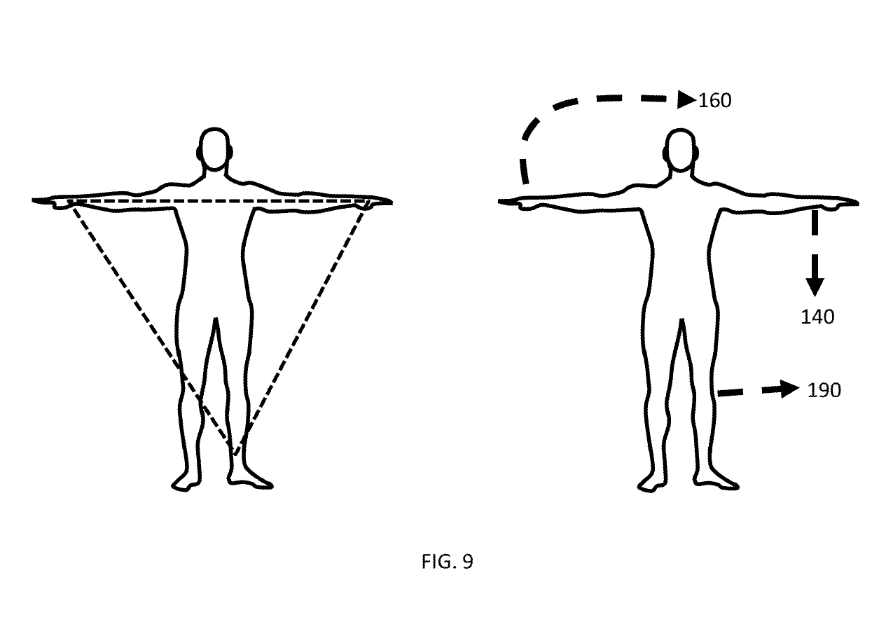

With reference to FIG. 9, the left panel shows a diagrammatic representation of a human and the relative distance of the electrodes, forming an imaginary equilateral triangle (Einthoven's triangle), with vertices located on the right arm, left arm and left leg; the sides of the triangle represent each one of the bipolar leads. The right panel of the figure shows the electric connection of the limbs to the device 100. The right arm connects to the contralateral arm electrode 160, the left arm connects to the ipsilateral arm electrode 140, and the left leg connects to the leg electrode 190.

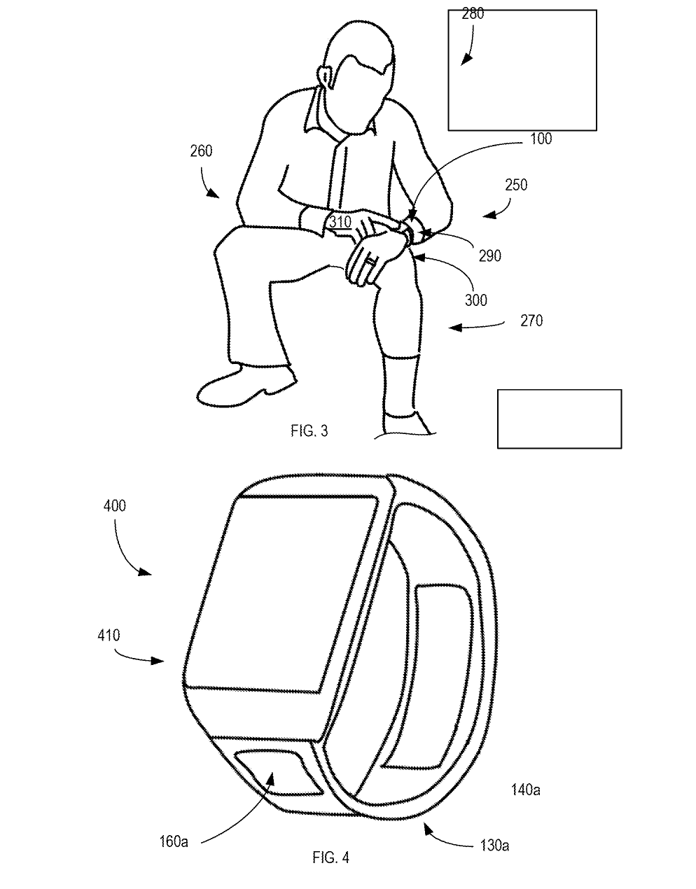

With reference additionally to FIG. 3, the hexaxial ECG recording device 100 will now be described in use. To record a hexaxial electrocardiogram, the ipsilateral arm electrode 140, the contralateral arm electrode 160, and the leg electrode 190 are placed into contact with (or receive contact from) a left arm 250, a right arm 260, and a left leg 270 of a user 280. As used herein, “arm” refers to any surface of user's arm from the tip(s) of the user's finger(s) to the anatomical region where the arm joins the thorax and leg refers to any surface of user's leg from the tip(s) of the user's toe(s) to the anatomical region where the leg joins the pelvis. In an alternative embodiment, the electrodes 140, 160, 190, are capable of making independent contact near the anatomical regions where the limb(s)/arm(s) attach to the thorax and/or the limb(s)/leg(s) attach to the pelvis.

With the user 280 in the shown sitting position and wearing the hexaxial ECG device 100 on a left wrist 290, and with a left pant leg rolled up, if necessary, to expose skin of a knee 300 of the user 280; the user 280 rests his left wrist 290 on his left knee 300 and touches the right arm (contralateral arm) electrode 160 with one's right hand 310.

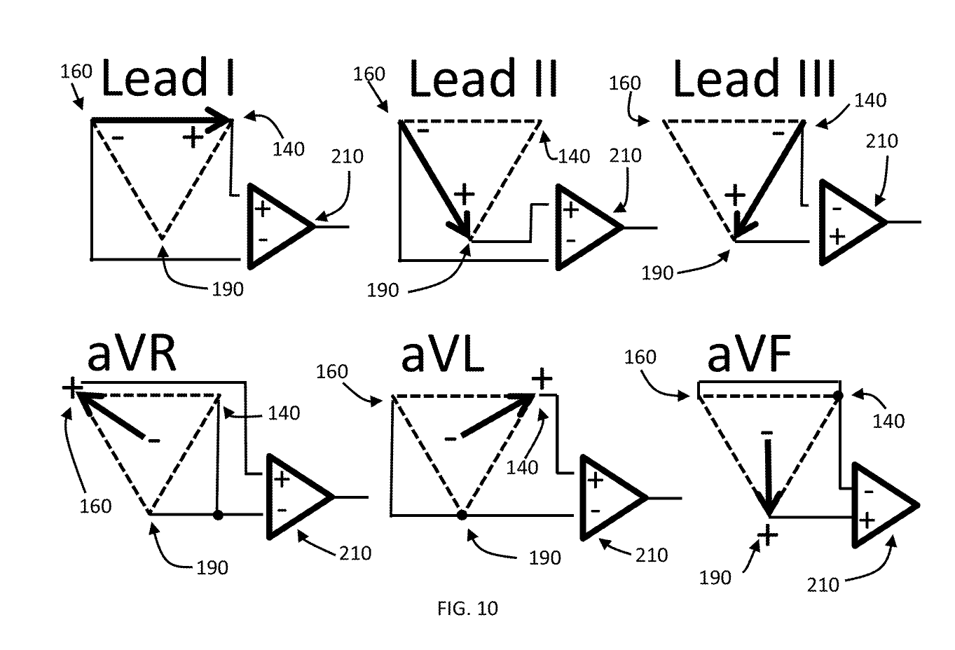

FIG. 10 illustrates the connectivity of electrodes 140, 160 and 190 shown in FIGS. 1-3 to obtain the hexaxial ECG. The upper left panel shows the connection to the right arm 260, through the contralateral electrode 160, to the inverting input of the amplifier 210 and the left arm 250, through the ipsilateral electrode 140, to the non-inverting input of the amplifier 210 to form bipolar lead I; the middle upper panel show the connection of the of the right arm 260, through the contralateral electrode 160, to the inverting input of the amplifier 210 and the left leg 270, through the leg electrode 190, to the non-inverting input of the amplifier 210 to form bipolar lead II; the upper right panel shows the connection of the left arm 250, through the ipsilateral electrode 140, to the inverting input of the amplifier 210 and the left leg 270, through the leg electrode 190, to the non-inverting input of the amplifier 210 to form bipolar lead III. The lower left panel shows the combined connection of the left arm 250, through the ipsilateral electrode 140, and the left leg 270, through the leg electrode 190, providing the Goldberger's central terminal for this lead, which is connected to the inverting input of the amplifier 210 and the right arm 260, through the contralateral electrode 160, to the non-inverting input of the amplifier 210 to form aVR; the middle lower panel shows the combined connection of the right arm 260, through the contralateral electrode 160, and the left leg 270, through the leg electrode 190, providing the Goldberger's central terminal for this lead, which is connected to the inverting input of the amplifier 210 and the left arm 250, through the ipsilateral electrode 140, to the non-inverting input of the amplifier 210 to form aVL; the lower right panel shows the combined connection of the right arm 260, through the contralateral electrode 160, and the left arm 250, through the ipsilateral electrode 140, providing the Goldberger's central terminal for this lead, which is connected to the inverting input of the amplifier 210 and the left leg 270, through the leg electrode 190, to the non-inverting input of the amplifier 210 to form aVF.

To obtain an electrocardiogram of a user, a controller is configured to sense an electric potential between the left arm 250 and the right arm 260 of the user 280 with one or more of the contralateral arm electrode 160, the ipsilateral arm electrode 140, and the leg electrode 190; sense an electric potential between the right arm 260 and the left leg 270 of the user 280 with one or more of the contralateral arm electrode 160, the ipsilateral arm electrode 140, and the leg electrode 190; sense an electric potential between the left arm 250 and the left leg 190 of the user 280 with one or more of the contralateral arm electrode 160, the ipsilateral arm electrode 140, and the leg electrode 270; sense the electric potential between the left arm 250 and a common electric point, representative of a central terminal with one or more of the contralateral arm electrode 160, the ipsilateral arm electrode 140, and the leg electrode 270; sense the electric potential between the right arm 260 and a common electric point, representative of a central terminal, with one or more of the contralateral arm electrode 160, the ipsilateral arm electrode 140, and the leg electrode 270; sense the electric potential between the left leg 270 and a common electric point, representative of a central terminal, with one or more of the contralateral arm electrode 160, the ipsilateral arm electrode 140, and the leg electrode 270; and determine one or more components of a frontal plane, six-lead electrocardiogram of the user 280 based on resulting signals of the sensed electric potentials. The frontal plane six-lead, or hexaxial, electrocardiogram is determined by the combined measurement of: bipolar lead I (L I=LA−RA); bipolar lead II (L II=LL−RA); bipolar lead III (L III=LL−LA); unipolar lead VR (VR=RA−Wilson's terminal), or aVR (aVR=RA−½[LA+LL], where ½[LA+LL] is the Goldberger's terminal); unipolar lead VL (VL=LA−Wilson's terminal), or aVL (aVL=LA−½[RA+LL], where ½[RA+LL] is the Goldberger's terminal); and unipolar lead VF (VF=LL−Wilson's terminal), or aVF (aVF=LL−½[RA+LA], where ½[RA+LA] is the Goldberger's terminal).

In a right-wrist version/embodiment of the hexaxial ECG device 100, where the device 100 is worn on a right wrist of the user 280, the contralateral arm electrode 160 in FIG. 1 becomes the left arm electrode; the ipsilateral arm electrode 140 in FIG. 1 becomes the right arm electrode. With the user 280 in the sitting position and wearing the hexaxial ECG device 100 on the right wrist, and with the left pant leg rolled up, if necessary, to expose the skin of the knee 300; the user 280 rests the right wrist on the left knee 300 and touches the left arm/face plate electrode 160 with his left hand.

With reference to FIG. 4, in another embodiment of a hexaxial ECG device 400, where like components include the same reference number, but an “a” suffix, the electronic components (e.g., contralateral arm electrode 160a, ipsilateral arm electrode 140a, and leg electrode (same location as leg electrode 190 in FIG. 2)) of the device 100 described above with respect to FIGS. 1-3 are embedded inside of a wristband strap 130a. In this embodiment of the hexaxial ECG device 400, the wristband strap 130a incorporating the device 400 is worn with a regular watch, a smart watch, or any other kind of bracelet-like device. For example, but not by way of limitation, the wristband strap 130a incorporating the device 400 may couple with an Apple Watch brand smart watch (or other smart watch) 410, where the device 400 wirelessly (e.g., via Bluetooth wireless technology standard) or through a wired connection communicates with the smart watch 410 for exchanging data between the device 400 and the smart watch 410. In this embodiment or another embodiment, a downloadable software application (“app”) with module(s) that provides the requisite functionality for providing and/or displaying information related to the hexaxial ECG recording to a user may be downloaded to the smart watch 410.

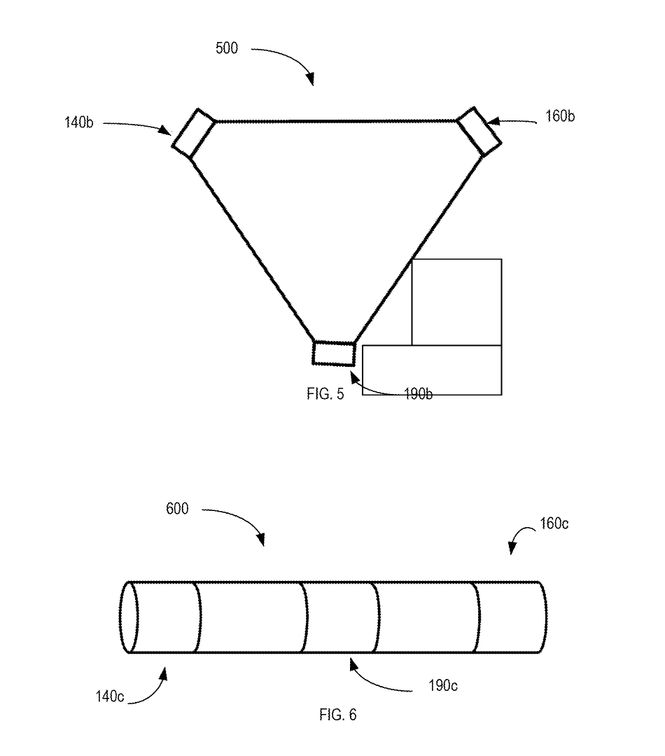

With reference to FIG. 5, in a further embodiment of a hexaxial ECG device 500, where like components include the same reference number, but a “b” suffix, the electronic components (e.g., contralateral arm electrode 160b, ipsilateral arm electrode 140b, and leg electrode 190b) of the device 100 described above with respect to FIGS. 1-3 are part of or in the form factor of a FOB small security hardware device.

With reference to FIG. 6, in a further embodiment of a hexaxial ECG device 600, where like components include the same reference number, but a “c” suffix, the electronic components (e.g., contralateral arm electrode 160c, ipsilateral arm electrode 140c, and leg electrode 190c) of the device 100 described above with respect to FIGS. 1-3 are part of or in the form factor of a wand.

In an additional embodiment, a hexaxial ECG device has the form factor of an arm chair. In this embodiment, the arm electrodes are placed on the arms of the chair and the leg electrode is located on the seat, in contact with the left leg of the subject.

In another embodiment, a hexaxial ECG device has the form factor of a driving wheel in an automobile. In this embodiment, the arm electrodes are located on the upper left and right regions of the driving wheel, and the leg electrode is located on the bottom part of the driving wheel.

In another embodiment, a hexaxial ECG device is part of, or is attached to, another electronic device such as, but not limited to, a computer, a laptop computer, a computer mouse, a cellular telephone, and a remote control.

In still further embodiments, the hexaxial ECG device has the form factor of one or more of the following: a patch, a device that is part of another device, a device that is mounted on another device, a cellular telephone, a walking stick, an anklet, a belt, a belt where the belt is part of an undergarment, a piece of furniture, a chair, bathroom furniture, and a toilet.

The hexaxial ECG recording devices 400, 500, 600 (and elsewhere described) are used in a similar manner to that described above with respect to FIG. 3. To record a hexaxial electrocardiogram, the ipsilateral arm electrode 140, the contralateral arm electrode 160, and the leg electrode 190 are placed into contact with (or receive contact from) a left arm 250, a right arm 260, and a left leg 270 of a user 280. The one or more components of a frontal plane, six-lead electrocardiogram of the user 280 are determined in a manner similar to that described above with respect to FIGS. 7, 9, and 10.

FIG. 8 is a block diagram illustrating an example wired or wireless device 550 that may be used in connection with various embodiments described herein. For example the device 550 may be used as or in conjunction with the hexaxial ECG recording devices/electronic circuit(s) as previously described with respect to FIGS. 1-7 and/or the smart watch 410. The device 550 can be a conventional personal computer, computer server, personal digital assistant, smart phone, smart watch, tablet computer, or any other processor enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include an internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily or exclusively in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein, and the controller as recited in the following claims, can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

In an embodiment of a controller for performing the steps described in connection with the embodiments disclosed, the controller includes a hybrid analog and digital circuit with a non-transitory computer readable medium configured to store executable programmed modules, a PIC processor coupled with the non-transitory computer readable medium configured to execute executable programmed modules stored therein, and computer programmed module elements stored in the non-transitory computer readable medium are configured to be executed by the PIC processor to perform the steps recited below).

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in any following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. A device for obtaining an electrocardiogram of a user, comprising:
a contralateral arm electrode for independent contact with one of a left arm and a right arm of the user;
an ipsilateral arm electrode for independent contact with an other of the left arm and the right arm of the user;
a leg electrode for independent contact with a leg of the user;
a controller configured to:
sense an electric potential between the left arm and the right arm of the user with one or more of the contralateral arm electrode, the ipsilateral arm electrode, and the leg electrode;
sense an electric potential between the right arm and the left leg of the user with one or more of the contralateral arm electrode, the ipsilateral arm electrode, and the leg electrode;
sense an electric potential between the left arm and the left leg of the user with one or more of the contralateral arm electrode, the ipsilateral arm electrode, and the leg electrode;
sense the electric potential between the left arm and a common electric point, representative of a central terminal with one or more of the contralateral arm electrode, the ipsilateral arm electrode, and the leg electrode;
sense the electric potential between the right arm and a common electric point, representative of a central terminal, with one or more of the contralateral arm electrode, the ipsilateral arm electrode, and the leg electrode;
sense the electric potential between the left leg and a common electric point, representative of a central terminal, with one or more of the contralateral arm electrode, the ipsilateral arm electrode, and the leg electrode;
determine one or more components of a frontal plane, six-lead electrocardiogram of the user based on resulting signals of the sensed electric potentials.

2. The device of claim 1, wherein the device is a wrist band.

3. The device of claim 1, wherein the device has a form factor of a fob.

4. The device of claim 1, wherein the device has a form factor of a wand.

5. The device of claim 1, wherein the device is one of a part of another device and mounted on another device.

6. The device of claim 1, wherein the device is part of a laptop computer.

7. The device of claim 1, wherein the device is part of a cellular telephone.

8. The device of claim 1, wherein the device includes memory for storing information.

9. The device of claim 1, wherein the device includes a wireless communication device.

10. The device of claim 9, wherein the wireless communication device is capable of transmitting a wireless signal to a cellular device.

11. The device of claim 10, wherein the cellular device is capable of displaying the signal.

12. The device of claim 10, wherein the cellular device is a cellular telephone.

13. The device of claim 1, wherein the controller includes a hybrid analog and digital circuit with a non-transitory computer readable medium configured to store executable programmed modules, a processor coupled with the non-transitory computer readable medium configured to execute executable programmed modules stored therein, and computer programmed module elements stored in the non-transitory computer readable medium and configured to be executed by the processor to perform the steps recited.

14. A device for obtaining an electrocardiogram of a user, comprising:
a first electrode for independent contact with an anatomical region where an arm of the user is attached to the thorax;
a second electrode for independent contact with an anatomical region where an other arm of the user is attached to the thorax;
a third electrode for independent contact with an anatomical region where a leg of the user is attached to the pelvis;
a controller configured to:
sense an electric potential between the left arm and the right arm of the user with one or more of the first electrode, the second electrode, and the third electrode;
sense an electric potential between the right arm and the left leg of the user with one or more of the first electrode, the second electrode, and the third electrode;
sense an electric potential between the left arm and the left leg of the user with one or more of the first electrode, the second electrode, and the third electrode;
sense the electric potential between the left arm and a common electric point, representative of a central terminal with one or more of the first electrode, the second electrode, and the third electrode;
sense the electric potential between the right arm and a common electric point, representative of a central terminal, with one or more of the first electrode, the second electrode, and the third electrode;
sense the electric potential between the left leg and a common electric point, representative of a central terminal, with one or more of the first electrode, the second electrode, and the third electrode;
determine one or more components of a frontal plane, six-lead electrocardiogram of the user based on resulting signals of the sensed electric potentials.

15. A method of obtaining a frontal plane, six-lead electrocardiogram of a user, comprising:
sensing an electric potential between a left arm and a right arm of the user with one or more of a contralateral arm electrode, an ipsilateral arm electrode, and a leg electrode;
sensing an electric potential between the right arm and a left leg of the user with one or more of the contralateral arm electrode, the ipsilateral arm electrode, and the leg electrode;
sensing an electric potential between the left arm and the left leg of the user with one or more of the contralateral arm electrode, the ipsilateral arm electrode, and the leg electrode;
sensing the electric potential between the left arm and a common electric point, representative of a central terminal with one or more of the contralateral arm electrode, the ipsilateral arm electrode, and the leg electrode;
sensing the electric potential between the right arm and a common electric point, representative of a central terminal, with one or more of the contralateral arm electrode, the ipsilateral arm electrode, and the leg electrode;
sensing the electric potential between the left leg and a common electric point, representative of a central terminal, with one or more of the contralateral arm electrode, the ipsilateral arm electrode, and the leg electrode;
determining one or more components of a frontal plane, six-lead electrocardiogram of the user based on resulting signals of the sensed electric potentials.
